(19) United States
(12) Patent Application Publication
(10) Pub. No.: US 2008/0026062 A1
(43) Pub. Date: Jan. 31, 2008

(54) Pharmaceutical compositions including nano-sized active agent

(76) Inventors: Isaac Farr, Corvallis, OR (US); Leslie Rivera, Carolina, PR (US); Ricardo G. Diaz-Felipe, Camas, WA (US); Javier Valentin-Sivico, Aguadilla, PR (US); Saul Tirado, Isabela, PR (US); Iddys D. Figueroa, Dorado, PR (US); Kevin Michael Kane, Oakland Park, FL (US); Mirayda Aponte, Aguadilla, PR (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/496,873

(22) Filed: Jul. 31, 2006

(51) Int. Cl. A61K 9/14

(52) U.S. Cl. 424/486

(57) ABSTRACT

The present invention is directed to a particulate pharmaceutical composition. The particulate pharmaceutical composition can comprise a water-soluble or partially water-soluble polymer matrix; and a plurality of nano-sized particles of active agent which are sparingly water-soluble to water-insoluble dispersed in the water-soluble or partially water-soluble polymer matrix. The particulate pharmaceutical composition can be micronized or in the form of a film that can be rolled up. If micronized, the individual micron-sized particles can have a plurality of nano-sized particles present in the micron-sized particles.

### BACKGROUND OF THE INVENTION

Poor dissolution behavior is observed for many sparingly water-soluble to water-insoluble drugs, which limits their bioavailability. For example, drugs such as various steroids, cyclosporines, and glyburide present delivery challenges due to their poor aqueous solubility and slow dissolution rate. Such low solubility can often result in low bioavailability, particularly given limited transit times through the gastrointestinal tract. Generally, water insoluble drugs with a smaller particle size have a greater rate of disolution (rate at which a substance goes into solution) and therefore greater bioavailability. Generally, as the size of a collection of solid or semisolid particles is decreased, the exposed surface area of the material from which the particles are generated is greatly increased. Poor dissolution behavior is currently dealt with in several different ways, each requiring a significant amount of expert resources to produce drugs with a smaller particle size. The most popular methods include forming molecular solid dispersions by milling the drug particles to reduce their size, and using complicated precipitation processes.

Nano-sized particles can be desirable for increasing the bioavailability of certain sparingly water-soluble to water-insoluble pharmaceuticals because of their small particle size. However, working with nano-sized particles presents several difficulties, including Ostwald ripening, agglomeration, and handling problems. Ostwald ripening occurs when concentration gradients cause small precipitates to dissolve and larger precipitates to grow. Bulk nano-sized particles are susceptible to particle growth via Ostwald ripening. To prevent Ostwald ripening, a common practice is to resign to designing formulations with larger drug particles or design-in a physical separation to limit transport phenomenon. Nano-sized particles also have a high propensity to agglomerate because of electrostatic or other non-covalent attractive forces. The propensity to agglomerate is much stronger in nano-sized particles than with other materials because of their smaller size, and by extension, smaller mass. Agglomeration is typically combated by blending in powder material to physically separate particles or by coating particles to force a separation. Nano-sized particles are also difficult to handle because of their size, which requires specialized tools, filters, and processes. Manufacturers within the pharmaceutical industry are generally equipped for processing micron-sized particles on a routine basis. However, very few pharmaceutical manufacturers process nano-sized particulate materials without some type of carrier. Additionally, current methods of collecting nano-sized particulates of drugs are often inefficient and manually intensive because such methods require scraping glass and filtering large volumes of liquid.

### DETAILED DESCRIPTION OF THE PREFERRED EMBODIMENT(S)

Reference will now be made to exemplary embodiments, and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art having possession of this disclosure, are to be considered within the scope of the present invention.

In describing and claiming the present invention, the following terminology will be used in accordance with the definitions set forth below.

The singular forms “a,” “an,” and “the” include plural referents unless the context clearly dictates otherwise. Thus, for example, reference to “a polymer matrix” includes one or more of such materials, reference to “an excipient” includes reference to one or more of such additives, and reference to “the step of” includes reference to one or more of such steps.

As used herein, “active agent” and “drug,” can be used interchangeably to refer to an agent or substance that has measurable specified or selected physiologic activity when administered to a subject in a pharmaceutically significant or effective amount. The active agent can be a therapeutic, a prophylactic, or a diagnostic agent. These terms of art are well-known in the pharmaceutical and medicinal arts.

As used herein “prodrug” refers to a molecule that will convert into a drug or active agent (its commonly known pharmacological active form). Prodrugs themselves can also be pharmacologically active, and therefore are also expressly included within the definition of an “active agent” as recited above.

As used herein, “micron-sized” refers to objects having an average size ranging from 2 μm to 1000 μm, as measured by light-scattering methods, microscopy, or other appropriate methods.

As used herein, “nano-sized” refers to objects having an average size ranging from 10 nm to less than 2000 nm, as measured by light-scattering methods, microscopy, or other appropriate methods.

Aspects of the present invention are directed to a particulate pharmaceutical composition, and methods of making and using the composition. It is noted that compositions and methods of the present invention share certain commonalities and characteristics. Some of these characteristics will be discussed in the context of the compositions, and others will be described in the context of the methods. It should be noted that regardless of where discussed, each of the following descriptions applies generally to the particulate pharmaceutical compositions and methods for making and using a particulate pharmaceutical compositions.

According to one embodiment of the present invention, a particulate pharmaceutical composition can comprise a water-soluble or partially water-soluble polymer matrix; and a plurality of nano-sized particles of active agent which are sparingly water-soluble to water-insoluble dispersed in the water-soluble or partially water-soluble polymer matrix. The particulate pharmaceutical composition can be micronized or rolled up. If micronized, the individual micron-sized particles can have a plurality of nano-sized particles present in the micron-sized particles. If the product is rolled-up, such as when produced using a roll-to-roll process, this provides a convenient means of packaging, shipping, and configuration for further process, e.g., cutting into individual pieces for transdermal, transmucosal, or oral delivery; micronizing for oral or parenteral delivery, etc.

The nano-sized particles have a faster rate of solution and bioavailability compared to larger particles of the same active agent because of their small size. Because the particles are dispersed throughout the polymer matrix, they are physically separated in the water-soluble matrix. This separation limits transport mechanisms which can lead to larger particles via Ostwald ripening effects. Agglomeration can also be reduced because the polymer matrix material forces a separation between the particles. Additionally, the particulate pharmaceutical composition includes micron-sized particulates of the composition, which are easier to contain and handle compared to particles of nano-sized sparingly water-soluble to water-insoluble active agent alone, because standard processing techniques for micron-sized materials can be used.

According to another embodiment of the present invention, a method of making a pharmaceutical composition is provided. The method can include dissolving a sparingly water-soluble to water-insoluble active agent in a solvent system comprising at least one organic solvent to form a dissolved active agent; applying the dissolved active agent to a water-soluble polymer matrix; at least partially dissolving the polymer matrix to form a paste containing the active agent and the polymer matrix; and evaporating the organic solvent to form a nano-sized precipitate of the active agent, wherein the nano-sized precipitate is dispersed throughout the polymer matrix. In one embodiment, the step of micronizing the polymer matrix containing the nano-sized precipitate can be carried out to form micron-sized particulates. In another embodiment, the film can be configured for further processing, such as by rolling.

Additional steps can optionally be included in the method of making the pharmaceutical composition. For example, steps can be taken to change the form of the micron-sized powder. Changing the form of the micron-sized powder can be desirable for several reasons; for example, dispensing a certain effective amount of the active agent and making the particulate pharmaceutical composition easier to administer to a subject. One aspect of the present invention includes the step of enclosing the micron-sized powder into a capsule or pressing the micron-sized powder into a tablet.

The method of making the pharmaceutical composition can also include applying the dissolved active agent to a polymer matrix. The dissolved active agent can be applied to the water-soluble polymer matrix by any process known to those in the art, including but not limited to an ink-jetting process. Ink-jetting processes can apply very small volumes of the dissolved active agent to the polymer matrix with a high degree of accuracy and reproducibility.

An organic solvent in which the active agent can be dissolved can be used with this invention. Non-limiting examples of types of solvents that can be used as the organic solvent in the solvent system include alcohols, chlorinated solvents, ketones, and combinations thereof. Specific examples of useful alcohols include, without limitation, hexanol, pentanol, butanol, propanol, ethanol, methanol, and combinations thereof. Specific examples of useful chlorinated solvents include, without limitation, chloroform, methylene chloride, and combinations thereof. A specific example of a useful ketone can include, without limitation, acetone. One specific embodiment of the present invention includes a solvent system where the organic solvent includes chloroform, ethanol, methanol, acetone, acetonitrile, and combinations thereof.

There are many ways of accomplishing the step of at least partially dissolving the polymer matrix. In one embodiment of the method of making a pharmaceutical composition, the polymer matrix can initially be in the form of a film before being dissolved. Any compound capable of dissolving the polymer matrix and precipitating the active agent can be used. Non-limiting examples of compounds that can be used to dissolve the polymer matrix include water and alcohols, or combinations thereof. In one specific embodiment of the method of making the pharmaceutical composition, the step of dissolving the polymer matrix can be performed by placing water in contact with the polymer matrix. The compound used to dissolve the polymer matrix can be in the form of a liquid or a vapor, and the compound can be applied separately or as part of the solvent system. In one embodiment, the step of at least partially dissolving the polymer matrix can occur simultaneously with the step of applying the dissolved active agent.

According to yet another embodiment of the present invention, a method of using a particulate pharmaceutical composition is provided. The method can include administering to a subject a pharmaceutical composition, wherein individual micron-sized particulates of the composition comprise a plurality of particles of nano-sized sparingly water-soluble to water-insoluble active agent dispersed in a water-soluble or partially water-soluble polymer matrix. One embodiment includes administering the pharmaceutical composition to a subject that is a mammal. A more specific aspect of the present invention includes administering the pharmaceutical composition to subject that is human.

The micron particulates of the particulate pharmaceutical composition can be in various forms. Certain forms of the composition can be desirable for several reasons; for example, dispensing a certain effective amount of the active agent and making the particulate pharmaceutical composition easier to administer to a subject. One embodiment of the present invention includes micron particulates that are enclosed by a capsule or pressed into a tablet.

An active agent can be used in the particulate pharmaceutical composition. The active agent can include a therapeutic, prophylactic, or diagnostic agent. Non-limiting examples of active agents that can be used with the instant invention include amiodarone HCL, atorvastatin, candesartan, carvedilol, clopidogrel bisulfate, dipyridamole, eprosartan mensylate, felodipine, furosemide, isradipine, lovastatin, metolazone, propafenone HCL, quinapril, ramipril, simvastatin, trandolapril, valsartan, clozapine, entacapone, fluphenazine, fluvoxamine, imipramine, olanzapine, paroxetine, sertraline, triazolam, zaleplon, ziprasidone, acyclovir, amphotericin B, amprenavir, cefdinir, cefixime, ceftazidime, clarithromycin, didanosine, efavirenz, ganciclovir, itraconazole, melfloquine, norfloxacin, nystatin, ritonavir, saquinavir, tenofovir disoproxil fumarate, beclomethasone dipropionate, bosentan, budesonide, fexofenadine, flunisolide, fluticasone, loratadine, mometasone, salmeterol xinafoate, triamcinolone acetonide, zafirlukast, celecoxib, diclofenac sodium, dihydroergotamine mesylate, ergoloid mesylates, ergotamine tartrate, fentanyl citrate, nabumetone, azathioprine, carboplatin, cisplatin, cyclosporine, docetaxel, etoposide, flurouracil, irinotecan, letrozole, melphalan, mitotane, paclitaxel, pimecrolimus, sirolimus, tacrolimus, valrubicin, ethinyl estradiol, danazol, follotropin beta, medroxy-progesterone, methyl-testosterone, raloxifene HCL, sildenafil citrate, testosterone, calcitrol, dronabinol, famotidine, glyburide, isotretinoin, megestrol, modafinil, nimodipine, pioglitazone, propofol, thalidomide, betamethasone, triamcinolone, piroxicam, glimepiride, glipizide, digoxin, prednisolone, indomethacine, nadolol, fluconazol, cisapride, ibuprofen, acetaminophen, carbamazepine, nifedipine, ketoprofen, and derivatives, prodrugs, mixtures, and combinations thereof.

The active agent can be chosen based on certain characteristics. For example, the active agent can be sparingly water-soluble to water-insoluble. An active agent will be considered sparingly water-soluble if no more than 1 part of the active agent dissolves in 30 parts water by weight. An active agent will be considered water-insoluble if no more than 1 part of the active dissolves in 10,000 parts water by weight. Additionally, the active agent can be nano-sized once in solid form in accordance with embodiments of the present invention. In one specific embodiment of the present invention, the average size of the active agent is less than 2000 nm, less than 1500 nm, or even less than 1000 nm in size.

As briefly described previously, the particulate pharmaceutical composition can include a polymer matrix in which the active agent is dispersed. The polymer matrix should be wholly or partially dissolvable upon the addition of a liquid that is not a good solvent for the active agent, e.g., water in liquid or vapor form for water insoluble active agents. Non-limiting examples of polymer matrices that are suitable for use with the present invention include pullulan, polyethylene oxide, polyethylene glycol, block copolymers based on ethylene oxide and propylene oxide, polyvinyl pyrrolidone, cellulose ethers such as methylcellulose, hydroxypropylcellulose, hydroxyethylcellulose, hydroxylethylmethylcellulose, sodium carboxymethylcellulose, and aliphatic polyesters such as polylactide, poly(E-caprolactone), polyglycolide, poly(DL-lactide-co-glycolide), and combinations thereof. One specific embodiment of the present invention includes a polymer matrix that includes pullulan. Pullulan is readily dissolvable in water, and can carry water insoluble active agents or drugs. Thus, when contacted with water, pullulan can be readily dissolved leaving nano-sized particulates of active agent.

In accordance with embodiments of the present invention, the very small size, e.g., less than 2000 nm, of the active agent allows the particulate pharmaceutical composition to have a faster rate of disolution and greater bioavailability compared to larger particles of the same active agent. In one embodiment, the micron-sized particulates of the pharmaceutical composition can be substantially dissolvable in simulated intestinal fluid within about 65 minutes when stirred at 75 rpm. This fast dissolution rate enables a smaller dosage to be administered and therefore reduces the cost of producing the pharmaceutical composition. It is noted that though the formation of the particulates includes nano-sized active agent dispersed in a micron-sized polymer matrix, the composition can be fabricated so that both are dissolvable in certain other solvents or fluids. For example, the nano-sized active agent can be insoluble in water, the polymer matrix can be readily water soluble, and both can be dissolvable in stomach or intestinal fluid or whatever dissolution medium is physiologically relevant for the particular formulation.

In addition to the dissolvable polymer matrix and the nano-sized active agent dispersed therein (which is not dissolvable in the same solvent that the polymer matrix is dissolvable in), the particulate pharmaceutical composition can also include an excipient. Non-limiting types of excipients that can be used with the present invention include surfactants, polymers, binders, disintegrants, lubricants, antioxidants, preservatives, enteric coatings, release modifiers, colorants, taste masking agents and combinations thereof. Excipients can be chosen based on a desired characteristic or property. For example, surfactants often adhere to the surface of the nano-sized particles, and thus facilitate the dispersion of the nano-sized particles in the mixtures in which the nano-sized particles are formed, the medium in which the nano-sized particles are taken up for administration, and the medium into which the nano-sized particles are later delivered such as the gastrointestinal fluid Excipients such as polymers can be added for their molecular weight, polymer hydrophilicity, polymer inherent viscosity, etc. Addition of excipients such as release modifiers can change the dissolution properties of the particulate pharmaceutical composition from a fast-release to a controlled release platform.

The particulate pharmaceutical composition can be made in any manner known to those of skill in the art. For example, the composition can be produced using a roll-to-roll process. To use a roll-to-roll process, the polymer matrix could be introduced in the form of a film on an initial roll. The roll of polymer matrix film can be advanced along a platform where certain steps described in the method of making a particulate pharmaceutical composition can be carried out. After evaporating the solvent, e.g., drying, from the polymer matrix containing the nano-sized precipitate, the composition can be re-rolled onto a receiving roll. The receiving roll can then be micronized to form a particulate pharmaceutical composition. A roll-to-roll process can be desirable for transporting large quantities of the pharmaceutical composition because the receiving roll could be transported before micronization.

There are many variations on the roll-to-roll process that can also be used in making a particulate pharmaceutical composition. For instance, instead of collecting the polymer matrix containing the nano-sized precipitate onto a receiving roll, the dried composition could be micronized after solvent evaporation (immediately or thereafter). The particulate pharmaceutical composition could also be collected as film or turned into a paste, which is then enclosed into capsules or used as an injectible form of the active agent. Another alternative is to dry the composition into a film, which is then cut into edible strips or used in transdermal or transmucosal applications.

Micronization can be accomplished by any method which produces micron-sized particles. Non-limiting examples of methods of micronization include crushing, grinding, and milling. Grinding can be accomplished by using a coffee-bean grinder, or other grinder used by those of skill in the art. Milling can be accomplished by ball milling, air-jet milling, fluid energy milling, or other processes used by those of skill in the art.

### EXAMPLES

The following examples are given to illustrate embodiments of the present invention. It should be understood, however, that the invention is not to be limited to the specific conditions or details described in these examples.

### Example 1

Glyburide is completely dissolved in a solution of choloroform (12.2 vol %), ethanol (48.8 vol %), and water (39 vol %) at a concentration of 3.3 mg/mL to form a drug solution. The drug solution is then placed onto a film of pullulan where the chloroform and ethanol are allowed to evaporate. As these lower boiling point solvents evaporate, the glyburide precipitates in the form of nano-sized particles. The water that remains dissolves the pullulan to yield a paste containing the nano-sized glyburide particles. The paste is then lyophilized, dried under hard-vacuum, and ground to yield the final micron-size drug product of nano-sized particles dispersed throughout the micron-sized pullulan matrix. In another embodiment, rather than immediately grinding, the product can be rolled up, such as by a roll-to-roll method, for later processing, e.g., cutting into pieces, micronized, etc.

### Example 2

Glyburide is completely dissolved in a solution of ethanol (65 vol %) and water (35 vol %) at a concentration of 3.3 mg/mL to form a drug solution. The drug solution is then placed onto a film of pullulan and the ethanol is allowed to evaporate. As the lower boiling point ethanol evaporates, the glyburide precipitates in the form of nano-sized particles. The water that remains dissolves the pullulan to yield a paste containing the nano-sized drug particles. The paste is then lyophilized, dried under hard-vacuum, and ground to yield the final micron-size drug product of nano-sized particles dispersed throughout the micron-sized pullulan matrix. In another embodiment, rather than immediately grinding, the product can be rolled up, such as by a roll-to-roll method, for later processing, e.g., cutting into pieces, micronized, etc.

### Example 3

Glyburide is completely dissolved in a solution of acetone (65 vol %) and water (35 vol %) at a concentration of 3.3 mg/mL to form a drug solution. The drug solution is then placed onto a film of pullulan and the acetone is allowed to evaporate. As the lower boiling point acetone evaporates, the glyburide precipitates in the form of nano-sized particles. The water that remains dissolves the pullulan to yield a paste containing the nano-sized drug particles. The paste is then lyophilized, dried under hard-vacuum, and ground to yield the final micron-size drug product of nano-sized particles dispersed throughout the micron-sized pullulan. In another embodiment, rather than immediately grinding, the product can be rolled up, such as by a roll-to-roll method, for later processing, e.g., cutting into pieces, micronized, etc.

### Example 4

Glyburide is completely dissolved in a solution of chloroform (20 vol %) and ethanol (80 vol %) at a concentration of 3 mg/mL to form a drug solution. The drug solution is applied onto a film of pullulan. Water vapor is then applied to the film and the cholorform/ethanol solution is allowed to evaporate. As evaporation occurs, the glyburide precipitates in the form of nano-sized particles. The condensed water dissolves the pullulan to yield a paste containing the nano-sized drug particles. The paste is then dried and milled to form a micron-sized drug product of nano-sized particles dispersed throughout the micron-sized pullulan matrix. In another embodiment, rather than immediately grinding, the product can be rolled up, such as by a roll-to-roll method, for later processing, e.g., cutting into pieces, micronized, etc.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

What is claimed is:

1. A particulate pharmaceutical composition, comprising:
a water-soluble or partially water-soluble polymer matrix; and
a plurality of nano-sized particles of active agent which are sparingly water-soluble to water-insoluble dispersed in the water-soluble or partially water-soluble polymer matrix, wherein said particulate pharmaceutical composition is micronized, thereby forming individual micron-sized particles having a plurality of nano-sized particles present in the micron-sized particles.

2. The composition of claim 1, wherein the individual micron-sized particulates are enclosed by a capsule or are pressed into a tablet.

3. The composition of claim 1, wherein the active agent is selected from amiodarone HCL, atorvastatin, candesartan, carvedilol, clopidogrel bisulfate, dipyridamole, eprosartan mensylate, felodipine, furosemide, isradipine, lovastatin, metolazone, propafenone HCL, quinapril, ramipril, simvastatin, trandolapril, valsartan, clozapine, entacapone, fluphenazine, fluvoxamine, imipramine, olanzapine, paroxetine, sertraline, triazolam, zaleplon, ziprasidone, acyclovir, amphotericin B, amprenavir, cefdinir, cefixime, ceftazidime, clarithromycin, didanosine, efavirenz, ganciclovir, itraconazole, melfloquine, norfloxacin, nystatin, ritonavir, saquinavir, tenofovir disoproxil fumarate, beclomethasone dipropionate, bosentan, budesonide, fexofenadine, flunisolide, fluticasone, loratadine, mometasone, salmeterol xinafoate, triamcinolone acetonide, zafirlukast, celecoxib, diclofenac sodium, dihydroergotamine mesylate, ergoloid mesylates, ergotamine tartrate, fentanyl citrate, nabumetone, azathioprine, carboplatin, cisplatin, cyclosporine, docetaxel, etoposide, flurouracil, irinotecan, letrozole, melphalan, mitotane, paclitaxel, pimecrolimus, sirolimus, tacrolimus, valrubicin, ethinyl estradiol, danazol, follotropin beta, medroxy-progesterone, methyl-testosterone, raloxifene HCL, sildenafil citrate, testosterone, calcitrol, dronabinol, famotidine, glyburide, isotretinoin, megestrol, modafinil, nimodipine, pioglitazone, propofol, thalidomide, betamethasone, triamcinolone, piroxicam, glimepiride, glipizide, digoxin, prednisolone, indomethacine, nadolol, fluconazol, cisapride, ibuprofen, acetaminophen, carbamazepine, nifedipine, ketoprofen, and derivatives, prodrugs, and combinations thereof.

4. The composition of claim 1, wherein the average size of the nano-sized sparingly water-soluble to water-insoluble active agent is less than 1500 nm.

5. The composition of claim 1, wherein the water-soluble polymer matrix comprises pullulan, polyethylene oxide, polyethylene glycol, block copolymers based on ethylene oxide and propylene oxide, polyvinyl pyrrolidone, cellulose ethers such as methylcellulose, hydroxypropylcellulose, hydroxyethylcellulose, hydroxylethylmethylcellulose, sodium carboxymethylcellulose, and aliphatic polyesters such as polylactide, poly(E-caprolactone), polyglycolide, poly(DL-lactide-co-glycolide), and combinations thereof.

6. The composition of claim 1, wherein the water-soluble polymer matrix comprises pullulan.

7. The composition of claim 1, wherein the pharmaceutical composition is substantially dissolvable in simulated intestinal fluid in less than 65 minutes when stirred at 75 rpm.

8. A rolled pharmaceutical composition, comprising:
a water-soluble or partially water-soluble polymer matrix; and
a plurality of nano-sized particles of active agent which are sparingly water-soluble to water-insoluble dispersed in the water-soluble or partially water-soluble polymer matrix, wherein said pharmaceutical composition in the form of a rolled up film.

9. The composition of claim 8, wherein the rolled-up film is prepared by a roll-to-roll process.

10. The composition of claim 8, wherein the active agent is selected from amiodarone HCL, atorvastatin, candesartan, carvedilol, clopidogrel bisulfate, dipyridamole, eprosartan mensylate, felodipine, furosemide, isradipine, lovastatin, metolazone, propafenone HCL, quinapril, ramipril, simvastatin, trandolapril, valsartan, clozapine, entacapone, fluphenazine, fluvoxamine, imipramine, olanzapine, paroxetine, sertraline, triazolam, zaleplon, ziprasidone, acyclovir, amphotericin B, amprenavir, cefdinir, cefixime, ceftazidime, clarithromycin, didanosine, efavirenz, ganciclovir, itraconazole, melfloquine, norfloxacin, nystatin, ritonavir, saquinavir, tenofovir disoproxil fumarate, beclomethasone dipropionate, bosentan, budesonide, fexofenadine, flunisolide, fluticasone, loratadine, mometasone, salmeterol xinafoate, triamcinolone acetonide, zafirlukast, celecoxib, diclofenac sodium, dihydroergotamine mesylate, ergoloid mesylates, ergotamine tartrate, fentanyl citrate, nabumetone, azathioprine, carboplatin, cisplatin, cyclosporine, docetaxel, etoposide, flurouracil, irinotecan, letrozole, melphalan, mitotane, paclitaxel, pimecrolimus, sirolimus, tacrolimus, valrubicin, ethinyl estradiol, danazol, follotropin beta, medroxy-progesterone, methyl-testosterone, raloxifene HCL, sildenafil citrate, testosterone, calcitrol, dronabinol, famotidine, glyburide, isotretinoin, megestrol, modafinil, nimodipine, pioglitazone, propofol, thalidomide, betamethasone, triamcinolone, piroxicam, glimepiride, glipizide, digoxin, prednisolone, indomethacine, nadolol, fluconazol, cisapride, ibuprofen, acetaminophen, carbamazepine, nifedipine, ketoprofen, and derivatives, prodrugs, and combinations thereof.

11. The composition of claim 8, wherein the film of the rolled up film has a first side and a second side, wherein the active agent is more concentrated at the first side than the second side.

12. The composition of claim 8, wherein the water-soluble polymer matrix comprises pullulan, polyethylene oxide, polyethylene glycol, block copolymers based on ethylene oxide and propylene oxide, polyvinyl pyrrolidone, cellulose ethers such as methylcellulose, hydroxypropylcellulose, hydroxyethylcellulose, hydroxylethylmethylcellulose, sodium carboxymethylcellulose, and aliphatic polyesters such as polylactide, poly(E-caprolactone), polyglycolide, poly(DL-lactide-co-glycolide), and combinations thereof.

13. The composition of claim 8, wherein the water-soluble polymer matrix comprises pullulan.

14. The composition of claim 8, wherein the pharmaceutical composition is substantially dissolvable in simulated intestinal fluid in less than 65 minutes when stirred at 75 rpm.

15. A method of making a pharmaceutical composition, comprising:
a) dissolving a sparingly water-soluble to water-insoluble active agent in a solvent system comprising at least one organic solvent to form a dissolved active agent;
b) applying the dissolved active agent to a water-soluble polymer matrix;
c) at least partially dissolving the polymer matrix to form a paste containing the active agent and the polymer matrix;
d) evaporating the organic solvent to form a nano-sized precipitate of the active agent, wherein the nano-sized precipitate is dispersed in the polymer matrix; and
e) drying the polymer matrix containing the nano-sized precipitate.

16. The method of claim 15, further comprising the step of rolling up the polymer matrix containing the nano-sized precipitate.

17. The method of claim 15, further comprising the step of micronizing the polymer matrix containing the nano-sized precipitate to form micron-sized particulates, wherein the micron-sized particulates have a plurality of nano-sized particles of active agent present therein.

18. The method of claim 17, further comprising the step of enclosing the micron-sized particulates into a capsule or pressing the micron-sized particulates into a tablet.

19. The method of claim 15, wherein the active agent is selected from amiodarone HCL, atorvastatin, candesartan, carvedilol, clopidogrel bisulfate, dipyridamole, eprosartan mensylate, felodipine, furosemide, isradipine, lovastatin, metolazone, propafenone HCL, quinapril, ramipril, simvastatin, trandolapril, valsartan, clozapine, entacapone, fluphenazine, fluvoxamine, imipramine, olanzapine, paroxetine, sertraline, triazolam, zaleplon, ziprasidone, acyclovir, amphotericin B, amprenavir, cefdinir, cefixime, ceftazidime, clarithromycin, didanosine, efavirenz, ganciclovir, itraconazole, melfloquine, norfloxacin, nystatin, ritonavir, saquinavir, tenofovir disoproxil fumarate, beclomethasone dipropionate, bosentan, budesonide, fexofenadine, flunisolide, fluticasone, loratadine, mometasone, salmeterol xinafoate, triamcinolone acetonide, zafirlukast, celecoxib, diclofenac sodium, dihydroergotamine mesylate, ergoloid mesylates, ergotamine tartrate, fentanyl citrate, nabumetone, azathioprine, carboplatin, cisplatin, cyclosporine, docetaxel, etoposide, flurouracil, irinotecan, letrozole, melphalan, mitotane, paclitaxel, pimecrolimus, sirolimus, tacrolimus, valrubicin, ethinyl estradiol, danazol, follotropin beta, medroxy-progesterone, methyl-testosterone, raloxifene HCL, sildenafil citrate, testosterone, calcitrol, dronabinol, famotidine, glyburide, isotretinoin, megestrol, modafinil, nimodipine, pioglitazone, propofol, thalidomide, betamethasone, triamcinolone, piroxicam, glimepiride, glipizide, digoxin, prednisolone, indomethacine, nadolol, fluconazol, cisapride, ibuprofen, acetaminophen, carbamazepine, nifedipine, ketoprofen, and derivatives, prodrugs, mixtures, and combinations thereof.

20. The method of claim 15, wherein the step of applying the dissolved active agent is by a printing process.

21. The method of claim 20, wherein the printing process is an ink-jetting process.

22. The method of claim 20, wherein the printing process is an electrostatic printing process.

23. The method of claim 15, wherein the solvent system comprises an alcohol, a chlorinated solvent, a ketone, or combinations thereof.

24. The method of claim 15, wherein the solvent system comprises chloroform, ethanol, methanol, acetone, acetonitrile, or combinations thereof.

25. The method of claim 15, wherein the polymer matrix comprises pullulan, polyethylene oxide, polyethylene glycol, block copolymers based on ethylene oxide and propylene oxide, polyvinyl pyrrolidone, cellulose ethers such as methylcellulose, hydroxypropylcellulose, hydroxyethylcellulose, hydroxylethylmethylcellulose, sodium carboxymethylcellulose, and aliphatic polyesters such as polylactide, poly(E-caprolactone), polyglycolide, poly(DL-lactide-co-glycolide), and combinations thereof.

26. The method of claim 15, wherein the polymer matrix comprises pullulan.

27. The method of claim 15, wherein the polymer matrix is in the form of a film before being at least partially dissolved.

28. The method of claim 15, wherein the step of at least partially dissolving the polymer matrix is performed by placing water in contact with the polymer matrix.

29. The method of claim 15, wherein the step of at least partially dissolving the polymer matrix occurs simultaneously with the step of applying the dissolved active agent.

30. The pharmaceutical composition produced in accordance with the method of claim 15.

31. The pharmaceutical composition of claim 30, in a micronized form.

32. The pharmaceutical composition of claim 30, in a film form.

33. A method of using a particulate pharmaceutical composition, comprising administering to a subject the pharmaceutical composition of claim 1.

34. The method of claim 33, wherein the step of administering is by oral delivery.

35. The method of claim 33, wherein the subject is human.
